(19) United States
(12) Patent Application Publication
(10) Pub. No.: US 2025/0089991 A1
(43) Pub. Date: Mar. 20, 2025

(54) MEDICAL VISUALIZATION SYSTEM

(71) Applicant: AMBU A/S, Ballerup (DK)

(72) Inventor: Kaspar Mat MATTHISON-HANSEN, Helsingør (DK)

(73) Assignee: AMBU A/S, Ballerup (DK)

(21) Appl. No.: 18/883,345

(22) Filed: Sep. 12, 2024

(30) Foreign Application Priority Data
Sep. 14, 2023 (EP) 23197400.7

(51) Int. Cl. A61B 1/00; A61B 1/05

(52) U.S. Cl. CPC A61B 1/00048; A61B 1/00009; A61B 1/00066; A61B 1/05

(57) ABSTRACT

A medical visualization system including a video processing apparatus and an endoscope. The video processing apparatus is operable to receive image data from the endoscope and includes a processor adapted to receive the image data from the one or more endoscopes and cause a display to display a live representation of the image data. The system further includes a transmission component adapted to transmit the image data and/or the data representative of the image data to the video processing apparatus, the transmission component including a component coupling part adapted to couple with the device coupling part.

### CROSS-REFERENCE TO RELATED APPLICATIONS

This application claims priority from and the benefit of European Patent Application No. 23 197 400.7, filed Sep. 14, 2023; the disclosure of said application is incorporated by reference herein in its entirety.

### TECHNICAL FIELD

The present disclosure relates to a medical visualization system and elements thereof. Particularly, a medical visualization system comprising a medical visualization device, such as an endoscope, and a video processing apparatus.

### BACKGROUND

A visualization apparatus, such as an endoscope, laryngoscope, endotracheal tube, or tracheostomy tube, may be utilized to visually examine certain areas of the body of a patient, such as inside a body cavity of the patient. The body cavity may be comprised in the airways, the digestive tract, the intestines, etc. Visual examination is facilitated by a camera in the visualization visualization apparatus communicatively connected to a video processing apparatus communicatively connected with an integrated or remote display screen. Video output from the camera may displayed with the coupled display screen, thereby allowing an operator to control and navigate the visualization apparatus to inspect an area of interest.

Single-use endoscopes mitigate or eliminate the risk for cross-contamination and improve availability of such instruments where and when they are needed in addition to eliminating the time and cost involved to clean re-usable endoscopes. Studies have shown that the carbon footprint of maintaining and cleaning re-usable endoscopes may be equal to or greater than the carbon footprint of single-use endoscopes. The increasing concern for environmental impact and climate change has resulted in a focus on reducing the carbon footprint from single-use products.

A single-use endoscope is designed to be used during a medical procedure and then discarded. The single-use endoscope may comprise a handle, an insertion cord extending from the handle towards a distal end, a camera at the distal end, and a cable extending from the endoscope to the video processing apparatus. These components are inseparable from each other during use. When the single-use endoscope is discarded, these components are discarded.

It follows logically from the concept of disposability that reducing the amount of waste may reduce costs for purchasers of the devices as well as reducing environmental impact. The challenge is to find solutions reducing the amount of waste without compromising patient safety and medical personnels' ability to perform procedures efficiently and satisfactorily while at the same time keeping the manufacturing costs of the devices as low as possible.

### SUMMARY

It is an object of the present disclosure to provide a solution which at least improves the solutions of the prior art. Particularly, it is an object of the present disclosure to provide a medical visualization system and elements thereof.

An advantage of the present disclosure is provision of a visualization system that reduces waste, thereby reducing environmental impact. This advantage is also applicable for reusable devices. Since also reusable devices need replacement and the present disclosure provides that elements may be replaced individually, the advantage is available for single-use and also reusable visualization devices, such as endoscopes. Furthermore, the present disclosure provides advantageous procedures for assembling various parts of the system, in particular in ways reducing physical contact with reusable components. Further advantages of the present disclosure will become clear for the skilled person when reading the present description.

In one aspect, the present disclosure provides a reusable transmission component and a single-use endoscope connectable to the transmission component. The transmission component is separable from the endoscope and replaces the traditional cable. Thus, when the endoscope disclosed herein is discarded, a traditional cable is not discarded, reducing the carbon footprint of the system. Advantageously, in some variations the transmission component is hands-free separable from the endoscope by an operator during a medical procedure, thereby preventing potential contamination of the transmission component since the operator does not have to touch the transmission component to separate it from the endoscope.

In another aspect, the present disclosure provides a medical visualization system comprising such a transmission component and endoscope. The medical visualization system further comprises a video processing apparatus. The medical visualization system may further comprise a support system for the video processing apparatus and/or the transmission component.

In an embodiment according to the first aspect, the endoscope comprises a device coupling part and the transmission component comprises a component coupling part adapted to couple with the device coupling part. Such coupling may be performed during a medical procedure by an operator, without the operator having to touch the transmission component. The transmission component is adapted to transmit image data and/or data representative of the image data to the video processing apparatus.

Also disclosed is a cable reel and a support bracket for a medical visualization system, such as the disclosed medical visualization system. The cable reel and/or the support bracket may separately or collectively also form part of the disclosed visualization system.

The transmission component may comprise a component processor. The component processor may be adapted to receive the image data from the camera module. The component processor may be further adapted to preprocess the image data to provide the data representative of the image data, which may be received by the video processing apparatus, such as the processor of the video processing apparatus. Thus, the transmission component may transmit the data representative of the image data to the video processing apparatus, such as to the processor of the video processing apparatus.

The component coupling part may comprise the component processor. Providing the component processor as part of the component coupling part may be advantageous so as to reduce the distance between the component processor and the camera module of the endoscope. Thereby, the signal between the camera module and the component processor travels a short distance, e.g. in comparison with the component processor being arranged in an opposite end of the transmission component or in the video processing apparatus. A short distance between the camera module and the component processor is beneficial to avoid and/or reduce noise in the signal.

The transmission component may transmit data and/or signals from the video processing apparatus to the endoscope, such as to the camera module and/or to the light emitter of the endoscope. For example, camera configuration data and/or light configuration data may be transmitted from the video processing apparatus to the endoscope/camera module/light emitter.

The component coupling part may comprise one or more physical activatable input mechanisms. The one or more physical activatable input mechanism may be or include one or more buttons, one or more levers, one or more switches or other suitable components known in the art. Activation of the one or more physical activatable input mechanisms may cause the transmission component to transmit one or more input mechanism signals to the video processing apparatus indicative of activation of the one or more physical activatable input mechanisms. Such input mechanism signals may activate various functions of the video processing apparatus, e.g. capturing still images, starting/stopping video recording, changing predefined settings, etc.

The handle of the endoscope may comprise one or more communicator elements, e.g. plungers, levers etc. The one or more communicator elements may be adapted to activate the one or more physical activatable input mechanisms upon being manipulated by a user. Accordingly, a user may activate the one or more physical activatable input mechanisms (and activate a corresponding function of the video processing apparatus) by manipulating a respective communicator element. Thereby, the parts being touched by the user may be simple, non-electronic elements, which may be provided as part of a disposable element, e.g. the endoscope. In this way, the risk of cross contamination may be reduced in a cost-efficient manner.

For example, a first communicator element of the one or more communicator elements may extend from a first side to a second side. The first side may be arranged exteriorly of the handle. The second side may be arranged interiorly of the handle. The second side may be adapted to contact a first physical activatable input mechanism of the one or more physical activatable input mechanism when the component coupling part is coupled with the device coupling part. A second communicator element of the one or more communicator elements may extend from a first side to a second side. The second side of the second communicator element may be adapted to contact a second physical activatable input mechanism of the one or more physical activatable input mechanism when the component coupling part is coupled with the device coupling part.

The transmission component may comprise a device wireless transceiver. The video processing apparatus may comprise an apparatus wireless transceiver. The device wireless transceiver may be a transmitter and/or a receiver. The apparatus wireless transceiver may be a receiver and/or a transmitter. The device wireless transceiver may be adapted to wirelessly communicate with the apparatus wireless transceiver. The apparatus wireless transceiver may be adapted to wirelessly communicate with the device wireless transceiver. The device wireless transceiver may be adapted to wirelessly transmit the image data and/or the data representative of the image data to the apparatus wireless transceiver. The apparatus wireless transceiver may be adapted to wirelessly receive the image data and/or the data representative of the image data from the device wireless transceiver. The apparatus wireless transceiver may be adapted to wirelessly transmit data and/or signals, e.g. camera configuration data and/or light configuration data, to the device wireless transceiver. The device wireless transceiver may be adapted to wirelessly receive data and/or signals from the apparatus wireless transceiver.

The transmission component may comprise a device cable. The device cable may be provided alternative to the device wireless transceiver or in addition to the device wireless transceiver. The device cable may extend from a first cable end to a second cable end. The component coupling part may be provided at the second cable end. The first cable end may be connectable to the video processing apparatus. A connector, e.g. connectable to the video processing apparatus, may be provided at the first cable end.

The device cable may comprise a stiffened portion. The stiffened portion may extend from the second cable end and/or from the component coupling part towards the first cable end. The stiffened portion may have a length of between 20-150 mm from the second cable end, such as between 40-100 mm, such as between 60-80 mm.

The stiffened portion may be straight or substantially straight. Alternatively, the stiffened portion may be curved, e.g. upwards and/or away from the operator.

The handle may be an elongate element extending along a handle axis. The stiffened portion of the device cable may extend in a normal plane normal to the handle axis. The stiffened portion may extend at an angle relative to the normal plane and/or the stiffened portion may be curved upwards or downwards relative to the normal plane. The stiffened portion may extend between an upper plane and a lower plane. The upper plane may form an angle of less than +15 degrees relative to the normal plane. The lower plane may form an angle of less than-15 degrees relative to the normal plane. The lower plane may form an angle of 30 degrees relative to the upper plane. The lower plane, the upper plane and the normal plane may intersect the handle axis at a common point.

The handle may have a front side and a back side. The front side may be opposite the back side. The front side may be facing in a forward direction. The back side may be facing in a backward direction. The forward direction may be opposite the backward direction. The handle may be adapted to be gripped by a first hand of an operator, e.g. such that the back side of the handle is facing in the backwards direction towards the wrist of the first hand, and/or such that the front side is facing in the forward direction opposite the backward direction, i.e. away from the wrist of the first hand. The stiffened portion may extend in a cable direction. The cable direction may be between the forward direction and the backwards direction. For example, an angle between the cable direction and the forward direction may be between 5 and 85 degrees, such as between 40 degrees and 65 degrees, e.g. 60 degrees or 45 degrees.

As mentioned above, the present disclosure also comprises a cable reel for a medical visualization system, such as the disclosed medical visualization system. Accordingly, the medical visualization system may comprise the cable reel. The cable reel may be adapted to reel in and/or unreel at least a portion of the device cable.

The cable reel may be driven by an actuator, such as a rotary actuator, e.g. an electrical motor, e.g. a DC motor. The actuator may drive the cable reel such that the portion of the device cable is reeled in and out as appropriate. Such actuated drive of the cable reel may be advantageous for reducing influence of the cable reel on the operator's ability to control the endoscope. The actuator may be activated to reel in the portion of the device cable based on a first control input. The actuator may be activated to unreel the portion of the device cable based on a second control input. The first control input and/or the second control input may, for example, be caused by a push button, a switch, or similar. Alternatively or additionally, the first control input and/or the second control input may be generated by the video processing apparatus, e.g. based on a user input at the video processing apparatus. Alternatively or additionally, the first control input and/or the second control input may be generated in response to the component coupling part being received in and/or removed from a coupling part retainer.

The cable reel may comprise a cable reel housing. The cable reel housing may enclose the portion of the device cable when reeled in. The cable reel housing may comprise an opening. The device cable may extend through the opening. The opening may be covered with bristles or similar. The cable reel housing may be opaque, such as opaque to UV light.

The cable reel may comprise a spool. The spool may be accommodated in the cable reel housing. The portion of the device cable may be rolled onto the spool when reeled in by the cable reel. The spool may be transparent, such as transparent to UV light. For example, the spool may be made from Polymethylmethacrylate (PMMA).

The cable reel may comprise a UV light emitter and/or a plurality of UV light emitters. The UV light emitter(s) may be provided for disinfection, such as disinfection of the transmission component and/or parts thereof, such as the device cable or part thereof. The UV light emitter and/or the plurality of UV light emitters may be accommodated in the cable reel housing. In some examples, the UV light emitter may be arranged inside the spool. The UV light emitter and/or the plurality of UV light emitters may be activated based on whether the portion of the device cable is rolled onto the spool. For example, the UV light emitter and/or the plurality of UV light emitters may be activated in response to and/or after the portion of the device cable has been reeled in by the cable reel, e.g. in response to and/or after the portion of the device cable has been reeled onto the spool.

The transmission component may comprise a counter. The counter may be indicative of the number of times the transmission component has been used and/or may be indicative of deterioration of the transmission component and/or parts thereof. For example, the counter may be indicative of deterioration of the device cable and/or the component coupling part. The counter may be an electronically readable counter, e.g. a flash memory or other suitable electronic memory known in the art. Alternatively or additionally, the counter may be a physical wear indicator, e.g. an indicator changing color over time and/or based on UV light exposure.

The transmission component, such as the device cable and/or the component coupling part, may be coated with silver. Thereby, an antimicrobial effect may be achieved. Using a silver coating may be advantageous both independently as well as in combination with UV disinfection as described above. Furthermore, silver coating may be beneficial for the transmission component both when utilizing wireless communication with the video processing apparatus as well as when utilizing wired communication using the device cable.

The component coupling part of the transmission component may have a primary coupling element. The primary coupling element may be elongated and/or may extend along a coupling part axis. The coupling part axis may be perpendicular to the cable direction, as mentioned above. The device coupling part of the endoscope may have a secondary coupling element. The secondary coupling element may form a cavity, e.g. along the handle axis. The secondary coupling element, such as the cavity of the secondary coupling element, may be adapted to receive the primary coupling element, e.g. by a linear movement along the coupling part axis and/or the handle axis.

The device coupling part, such as the secondary coupling element, may comprise a twist lock. The twist lock may be adapted to engage by rotation of the handle with a protruding element of the component coupling part, e.g. of the primary coupling element, when the primary coupling element is fully received by the secondary coupling element. The rotation of the handle may be around the coupling part axis and/or the handle axis. The protruding element of the component coupling part may be the stiffened portion of the device cable.

The device coupling part, such as the secondary coupling element, may comprise one or more device terminals. The one or more device terminals may be electronically connected to the camera module and/or the light emitter. The component coupling part, such as the primary coupling element may comprise one or more component terminals. The one or more component terminals may be adapted to connect to the one or more device terminals of the device coupling part when the component coupling part is coupled with the device coupling part. Thereby, an electrical connection may be obtained between the camera module and/or the light emitter and the transmission component, such as between the camera module and/or the light emitter and the component processor of the transmission component.

As mentioned above, the present disclosure also comprises a support bracket for a medical visualization system, such as the disclosed medical visualization system. Accordingly, the medical visualization system may comprise the support bracket. The support bracket may have a distal support bracket part and a proximal support bracket part. The support bracket may extend between the distal support bracket part and the proximal support bracket part. The distal support bracket part may include a coupling part retainer. The coupling part retainer may be adapted to retain the component coupling part of the transmission component. In some examples, the support bracket may comprise the cable reel.

The coupling part retainer may comprise a switch, e.g. a mechanical switch (e.g. a button) or a hall switch. The switch of the coupling part retainer may be activated/deactivated based on the receipt of the component coupling part. The switch of the coupling part retainer may cause activation of the cable reel, such as to reel in or unreal the portion of the device cable. For example, the switch of the coupling part retainer may generate the first control input to the actuator of the cable reel to reel in the portion of the device cable, e.g. when the component coupling part is received in the coupling part retainer and/or the switch of the coupling part retainer may generate the second control input to the actuator of the cable reel to unreel the portion of the device cable, e.g. when the component coupling part is not received in and/or is removed from the coupling part retainer. In other words, the first control input may be triggered by the component coupling part being received by the coupling part retainer, e.g. the switch of the coupling part retainer may be subjective to receipt of the component coupling part and the switch may trigger the first control input. Additionally or alternatively, the second control input may be triggered by the component coupling part being removed from the coupling part retainer, e.g. the switch of the coupling part retainer may be subjective to removal of the component coupling part and the switch may trigger the second control input.

The proximal support bracket part may be adapted to be coupled to the video processing apparatus, such as to the housing of the video processing apparatus. For example, the proximal support bracket part may be adapted to be coupled to the video processing apparatus, such as to the housing of the video processing apparatus, such that the support bracket follows rotation of the housing around a first axis and/or such that the support bracket does not follow rotation of the housing around a second axis. The second axis may be perpendicular to the first axis. The first axis may be vertical. The second axis may be horizontal.

The video processing apparatus may comprise a mounting assembly. The mounting assembly may be adapted to attach the video processing apparatus, such as the housing of the video processing apparatus, to a mount, e.g. a stand, such as an IV pole. The mounting assembly may have a fastener, such as a clamp or similar. The fastener may be adapted to attach the video processing apparatus, such as the housing of the video processing apparatus, to the mount. The mounting assembly may have first hinge and/or a second hinge. The first hinge may be rotatable around a first axis (e.g. the same first axis as mentioned in the previous paragraph). The second hinge may be rotatable around a second axis (e.g. the same second axis as mentioned in the previous paragraph). The second axis may be perpendicular to the first axis. The first axis may be vertical. The second axis may be horizontal. The first hinge may be arranged between the fastener and the second hinge. The second hinge may be arranged between the first hinge and the housing of the video processing apparatus. The proximal support bracket part may be coupled to the mounting assembly between the first hinge and the second hinge.

The coupling part retainer may be adapted to receive and/or supply the component coupling part by linear movement of the component coupling part along a third axis. The component coupling part may be adapted to engage with and/or be removed from the coupling part retainer by linear movement of the component coupling part along the third axis. The third axis may be perpendicular to the first and/or second axis of any of the preceding paragraphs.

The coupling part axis may be non-parallel with the third axis, when the component coupling part is received by the coupling part retainer. For example, the third axis and the coupling part axis may form an angle, such as an angle between 45-90 degrees. For example, the third axis and the coupling part axis may be perpendicular.

The coupling part retainer and/or the component coupling part may be adapted to prevent rotation, e.g. at least around the first axis, the second axis and/or the third axis, of the component coupling part, e.g. relative to the coupling part retainer, when the component coupling part is received by the coupling part retainer.

The coupling part retainer and/or the component coupling part may be adapted to prevent movement, such as linear movement of the component coupling part, along the first axis and/or the second axis, when the component coupling part is received by the coupling part retainer.

The support bracket may comprise a support cable. The support cable may be extending between the distal support bracket part and the proximal support bracket part. The support cable may have a first end. The first end of the support cable may be arranged at the proximal support bracket part. The first end of the support cable may be adapted to connect to the video processing apparatus. For example, the first end of the support cable may comprise a connector. The support cable may have a second end. The support cable may extend from the first end to the second end. The second end of the support cable may be arranged at the distal support bracket part. The second end of the support cable may be adapted to connect to the transmission component. The support cable may be an integrated cable. For example, the support cable may be extending inside the support bracket.

The endoscope may be a laryngoscope, a broncoscope, a duodenoscope, a urology endoscope, or other type of endoscope.

The endoscope may comprise an insertion cord extending from the handle to a distal cord portion. The view from the endoscope may be a view from the distal cord portion of the insertion cord. The handle may comprise a control mechanism adapted to receive an input in a first input direction. The input in the first input direction may cause a bendable section of the insertion cord to bend in a first bending direction. The control mechanism may be adapted to receive an input in a second input direction, e.g. opposite the first input direction. The input in the second input direction may cause the bendable section of the insertion cord to bend in a second bending direction, e.g. opposite the first bending direction. In some examples, the control mechanism may be adapted to receive an input in a third input direction and/or to receive an input in fourth input direction, which respectively may cause the bendable section of the insertion cord to bend in a third and fourth bending direction, e.g. perpendicular to the first and/or second bending direction. Alternatively, the endoscope may comprise a second control mechanism adapted to receive the input in the third and/or fourth input directions.

The camera module of the endoscope may comprise an image sensor, such as a charge-coupled device (CCD) image sensor or a complementary metal-oxide-semiconductor (CMOS) image sensor, to generate the image data indicative of the view from the endoscope. The endoscope may further comprise a light emitter, e.g. adapted to provide illumination of the view. The light emitter may be an LED, an optical fiber connectable to a light source, or similar element known to provide illumination. The camera module may include the light emitter. The camera module and/or the light emitter may be provided at a distal end of an insertion cord of the endoscope.

The endoscope may be disposable. The transmission component may be reusable. The transmission component may be adapted to be coupled to a plurality of endoscopes, such as a plurality of different types of endoscopes.

The video processing apparatus may comprise the display. The display may be accommodated in the housing of the video processing apparatus. Alternatively or additionally, the display may be an external display coupled to the video processing apparatus, such as to allow the video processing apparatus to cause it to display the live representation of the image data.

### BRIEF DESCRIPTION OF THE FIGURES

Embodiments of the disclosure will be described in more detail in the following with regard to the accompanying figures. The figures are not to be construed as being limiting to other possible embodiments falling within the scope of the disclosure.

FIG. 1A schematically illustrates an embodiment of medical visualization system including an endoscope and a video processing apparatus;

FIG. 1B is a block diagram schematically illustrating the embodiment of the video processing apparatus of FIG. 1;

FIG. 2A is a block diagram schematically illustrating an embodiment of a transmission component;

FIG. 2B is a block diagram schematically illustrating a variation of the embodiment of the endoscope of FIG. 1;

FIG. 3A is a block diagram schematically illustrating a variation of the embodiment of the transmission component of FIG. 2A;

FIG. 3B is a block diagram schematically illustrating a variation of the embodiment of the endoscope of FIG. 1;

FIG. 4A schematically illustrates an embodiment of a component coupling part;

FIG. 4B schematically illustrates a variation of a portion of the embodiment of the endoscope of FIG. 1;

FIG. 5A schematically illustrates an exploded view of a variation of the embodiment of the component coupling part of FIG. 4A and of the embodiment of the endoscope of FIG. 4B;

FIG. 5B schematically illustrates an unexploded view of the variation of the embodiment of the component coupling part of FIG. 4A and of the embodiment of the endoscope of FIG. 4B;

FIG. 5C schematically illustrates a variation of FIG. 5B showing a user's hand;

FIG. 5D schematically illustrates a detailed view of FIG. 5B;

FIG. 6 schematically illustrates a top view of the variation of the embodiment of the transmission component of FIG. 5A and of the embodiment of the endoscope of FIG. 5B;

FIG. 7 schematically illustrates an embodiment of a mounting assembly;

FIG. 8 schematically illustrates an embodiment of a support bracket and a component coupling part;

FIG. 9 schematically illustrates a variation of the medical visualization system of FIG. 1;

FIG. 10A schematically illustrates another variation of the medical visualization system of FIG. 1;

FIG. 10B schematically illustrates internal parts of an embodiment of a cable reel;

FIGS. 11A, 11B, and 12 schematically illustrate control circuits to control the cable reel of FIG. 10B.

### DETAILED DESCRIPTION

Various embodiments and details are described hereinafter with reference to the figures when relevant. The figures may or may not be drawn to scale and elements of similar structures or functions are represented by like reference numerals throughout the figures. The figures are only intended to facilitate the description of the embodiments, they are not intended as an exhaustive description of the invention or as a limitation on the scope of the invention. In addition, an illustrated embodiment need not have all the aspects or advantages shown. An aspect or an advantage described in conjunction with a particular embodiment is not necessarily limited to that embodiment and can be practiced in any other embodiment even if not so illustrated, or if not so explicitly described.

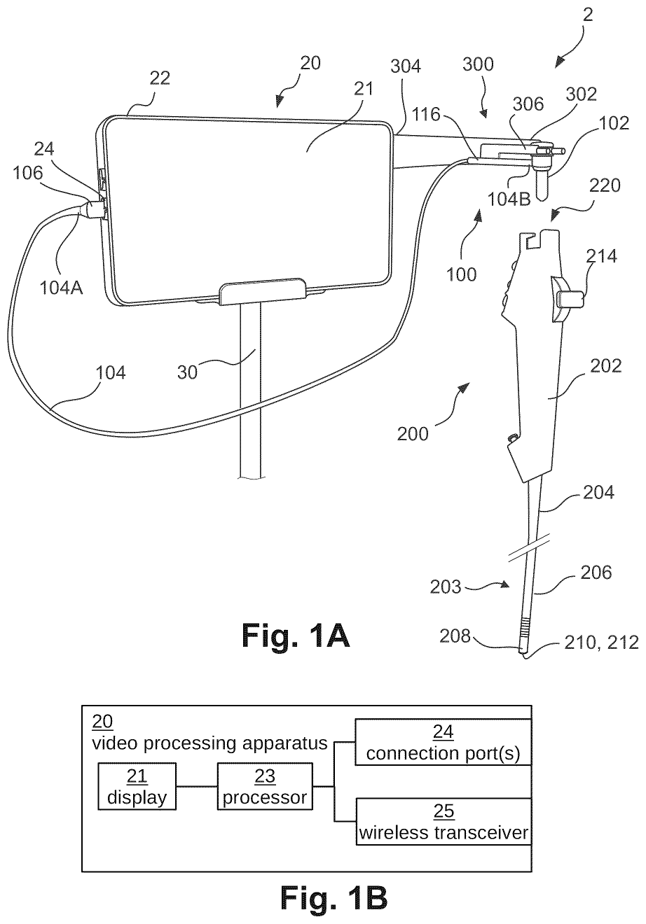

FIG. 1A schematically illustrates an embodiment of medical visualization system 2 comprising an endoscope 200 and a video processing apparatus 20. More generally, the medical visualization system 2 may comprise a visualization apparatus configured to be inserted into a patient (human or animal) and comprising an image sensor configured to capture internal views of the patient. Example visualization apparatus include endoscopes, endotracheal tubes, tracheostom tubes, laryngeal masks, laryngoscopes and cannulas. In the illustrated example, the endoscope 200 comprises a handle 202 and an insertion cord 203 extending distally from the handle 202. The insertion cord 203 comprises a insertion tube 204, a bending section 206, and a distal cord portion, or tip, 208. The tip 208 has a camera module 210, e.g. including an image sensor, such as a CCD or a CMOS image sensor, configured to generate image data indicative of a view from the endoscope 200. The camera module 210 may be adapted to generate image data indicative of a view from the tip 208 of the insertion cord 203, such as a distal view, a lateral view, or both distal and lateral views. The tip may further comprise a light emitter 212 adapted to provide illumination of the view. The light emitter 212 may be a light emitting diode (LED), an optical fiber connectable to a light source, or an analogous element known to provide illumination. In some examples, the light emitter 212 may form part of the camera module 210. The camera module 210 and the light emitter 212 may be enclosed in a tip housing attached to the bending section 206. More generally, the camera module 210 and the light emitter 212 are positioned at the distal end of the visualization apparatus, with or without a tip housing. The camera module 210 and the light emitter 212 may be positioned in a cavity of a wall of an endotracheal tube, for example. Embodiments of visualization apparatus are described in commonly-owned U.S. Pat. Nos. 9,486,595, 10,149,602, 10,478,054, 10,842,368, 11,109,745, U.S. Patent Publication No. 2023/0380676, and U.S. Patent Publication No. 2023/0397795, the disclosures of said patent and publication being incorporated herein by reference in their entirety. The visualization apparatus described in the patents and applications listed in the preceding sentence may be suitably adapted with a device coupling part 220, described below and adapted in the manner described below, to communicate with a video processing apparatus via a transmission component 100, also described below.

The visualization system 2 comprises a transmission component 100 adapted to transmit the image data and/or data representative of the image data to the video processing apparatus 20. Thereby, the endoscope 200 may be connected to the video processing apparatus 20. In the illustrated example, the transmission component 100 comprises a device cable 104. The device cable 104 extends from a first cable end 104A to a second cable end 104B. The device cable 104, in the illustrated example, terminates in a device connector 106 connected to a connection port 24 of the video processing apparatus 20. The device connector 106 is provided at the first cable end 104A. The device connector 106 may be a plug insertable into a receptacle, e.g. the connection port 24.

The transmission component 100 comprises a component coupling part 102, which is adapted to couple with the endoscope 200, such as with a device coupling part 220 of the endoscope. The component coupling part 102 is provided at the second cable end 104B. Thus, in the present example, the device cable 104 extends from the device connector 106 to the component coupling part 102. Thus, in the present example, when the component coupling part 102 is coupled with the device coupling part 220, the transmission component 100 extends from the handle 202 of the endoscope 200 to the connection port 24 of the video processing apparatus.

The transmission component 100, such as the device cable 104 and/or the component coupling part 102, may be coated with silver to obtain an antimicrobial surface.

The device cable 104 comprises a stiffened portion 116 extending from the second cable end 104B and/or from the component coupling part 102 towards the first cable end 104A. The stiffened portion may have a length of between 20-150 mm from the second cable end 104B.

The video processing apparatus 20 is operable to receive the image data generated by the camera module 210 of the endoscope 200. For example, the video processing apparatus 20 may receive image data generated by the camera module 210 via the device coupling part 220, the device coupling part 102, the device cable 104, the connector 106 and the connection port 24. Embodiments of video processing apparatus are described in commonly-owned European Patent Application No. 24169257.3, titled “NEAR TRUE-VIEW MEDICAL VIDEO PROCESSOR,” filed Apr. 9, 2024 and U.S. Pat. No. 11,109,741, titled “VIDEO PROCESSING APPARATUS,” the disclosures of said application and patent are incorporated herein by reference in their entirety.

The video processing apparatus 20 is configured to display images received from the endoscope 200. The video processing apparatus 20 comprises a housing 22. The video processing apparatus 20 may comprise a display 21, such as a touch sensitive display, as illustrated. Preferably, the display 21 is accommodated in the housing 22 of the video processing apparatus 20, as illustrated. Alternatively, or additionally, the video processing apparatus 20 may be connected to an external display where the images received from the endoscope 200 are displayed.

The handle 202 comprises a control, or steering, mechanism 214 adapted to receive an input (e.g. a touch input) in a first input direction and/or in a second input direction. The input in the first input direction on the steering mechanism 214 causes a bending section 206 of the insertion cord 203 to bend in a first bending direction, e.g. via wires extending from the handle 202, through the insertion cord 203 to the bending section 206. The input in the second input direction on the steering mechanism 214 causes the bending section 206 of the insertion cord 203 to bend in a second bending direction. The first input direction and the second input direction may be opposite. The first bending direction and the second bending direction may be opposite. Bending the bending section 206 of the insertion cord 203 may cause a movement of the tip 208 and the camera module 210 in a direction relative to the camera module 210. Thereby, seeing an image generated by the camera module 210, a direction, e.g. up or down, in the image may correspond to a respective input on the steering mechanism 214. In some examples, the endoscope 200 may additionally provide the possibility of also bending the bending section 206 in non-parallel direction, e.g. in a perpendicular direction. This additional non-parallel or perpendicular bending may be provided for by the steering mechanism 214 or by an additional control mechanism. Hence, the bending section may in such case cause movement of the tip 208 in four directions, e.g. left/right and up/down.

The endoscope 200 may be disposable, e.g. single use. Conversely, the transmission component 100 may be reusable. Thus, the transmission component 100 may be adapted to be coupled to a plurality of visualization apparatus, e.g. different types of endoscopes, e.g. including bronchoscopes, laryngoscopes, duodenoscopes, urology endoscopes etc.

The medical visualization system 2 further comprises an optional support bracket 300. The support bracket 300 has a distal support bracket part 302 and a proximal support bracket part 304. The support bracket 300 extends between the distal support bracket part 302 and the proximal support bracket part 304. The support bracket 300 comprises a coupling part retainer 306. The coupling part retainer 306 is adapted to retain the component coupling part 102, as illustrated. In the illustrated example, the distal support bracket part 302 includes the coupling part retainer 306. The proximal support bracket part 304 is coupled to video processing apparatus 20, such as the housing 22 of the video processing apparatus 20. In some examples, the support bracket 300 may follow rotation of the housing 22 around a first axis, e.g. a vertical axis, e.g. while not following rotation of the housing 22 around a second axis perpendicular to the first axis, e.g. a horizontal axis.

FIG. 1B is a block diagram schematically illustrating an embodiment of a video processing apparatus 20, such as the video processing apparatus 20 of FIG. 1A. The video processing apparatus 20 comprises a processor 23 adapted to receive the image data and/or data representative of the image data, e.g. via the connection port 24, as explained above. The processor 23 is further adapted to cause a display, e.g. the optional display 21 of the video processing apparatus 20, to display a live representation of the image data. The video processing apparatus 20 may also or alternatively comprise an apparatus wireless transceiver 25. Accordingly, the processor 23 may be adapted to receive the image data and/or data representative of the image data via the apparatus wireless transceiver 25, which will be explained in further detail below.

By “live representation” or “live view” it is meant that images or video are received by the video processing apparatus from the endoscope or visualization apparatus and presented with a display in substantially real-time. By “real-time” it is meant that the video processing apparatus processes the images/video generated by the visualization apparatus while the visualization apparatus generates them with minimal (in the order of milliseconds or less than 6 frames at 30 fps, preferably 3 or less frames) latency so that the physician observing the live view can rely on the view being representative of the current position of the visualization apparatus.

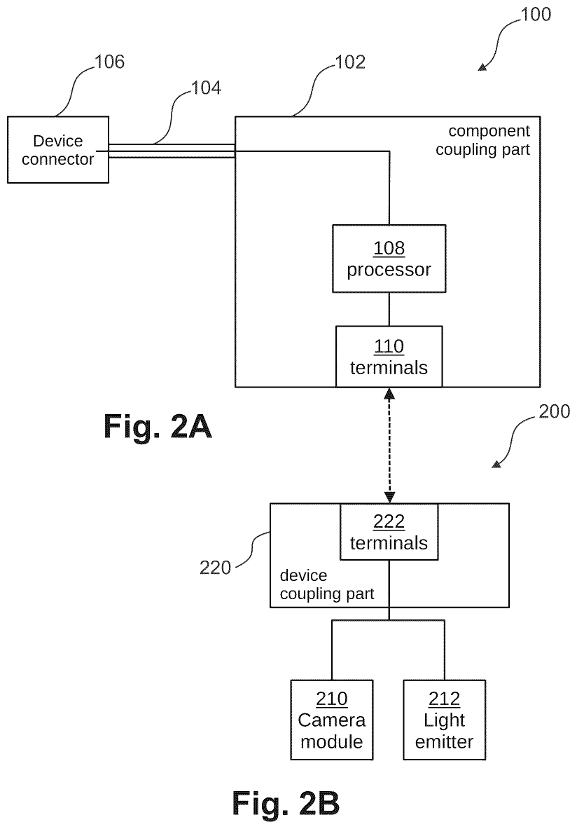

FIG. 2A is a block diagram schematically illustrating a variation of the embodiment of the transmission component 100 of FIG. 1, with the component coupling part 102, the device cable 104, and the device connector 106. FIG. 2B is a block diagram schematically illustrating a variation of the embodiment of the endoscope 200 of FIG. 1, with the camera module 210 and the light emitter 212.

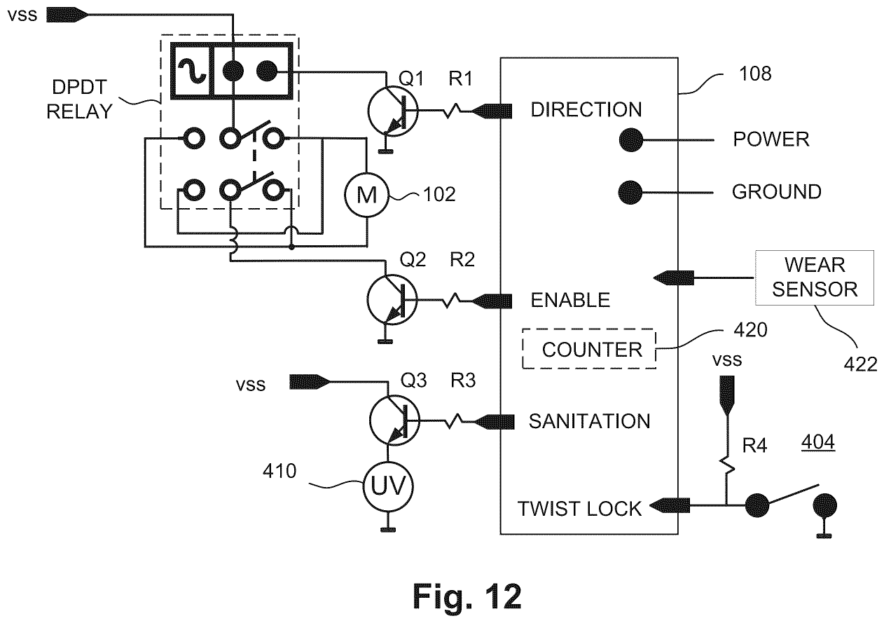

The transmission component 100 comprises a component processor 108, which is adapted to receive the image data from the camera module 210 and preprocess the image data to provide data representative of the image data, which may then be transmitted to the video processing apparatus, e.g. via the device cable 104 and the device connector 106. The component coupling part 102 may comprise the component processor 108, as illustrated. The component processor 108 may perform control functions (see FIG. 12) in addition to preprocessing image data. The processor may be a central processing unit (CPU), a field programmable gate array (FPGA), or a system-on-a-chip, for example. The visualization apparatus may comprise an image sensor in the camera that outputs image data via the mobile industry processor interface (MIPI) bridge standard. Data sent via the MIPI bridge is susceptible to noise. Pre-processing may comprise converting the image data in the MIPI standard to a different format so that it can be sent from the processor 108 to the video processing apparatus over the length of the cable 104 without errors. Data representative of the image data, in this context, means that the format of the image data is changed but the content, images, are substantially unaffected by the transformation to a different format, as is known in the art. Advantageously, by providing the processor 108 in the component coupling part 102, the processor 108 can be removed from the handle, which is single-use, and placed in a reusable component, reducing the carbon footprint and reducing costs.

The transmission component 100 also comprises one or more component terminals 110 adapted to connect to one or more device terminals 222 of the device coupling part 220 when the component coupling part 102 is coupled with the device coupling part 220. The component coupling part 102 may comprise the one or more component terminals 110. The component terminals 110 may form an electrical connection between the transmission component 100 and the endoscope 200, such as between the component coupling part 102 and the device coupling part 220. The component terminals 110 may be connected with the component processor 108 and may facilitate an electrical connection between the component processor 108 and the camera module 210 of the endoscope, i.e. facilitating the receipt of the image data generated by the camera module 210.

The device coupling part 220 is adapted to be coupled with the component coupling part 102. As shown, the device coupling part 220 comprises one or more device terminals 222 electrically connected to the camera module 210 and optionally to the light emitter 212. The device terminals 222 may facilitate an electrical connection between the component processor 108 of the transmission component 100 and the camera module 210, i.e. facilitating the transmission of the image data generated by the camera module 210.

In some examples, the transmission component 100 may facilitate transmission of data and/or signals from the video processing apparatus to the endoscope 200, such as to the camera module 210 and/or the light emitter 212. For example, camera configuration data may be transmitted from the video processing apparatus to the endoscope 200/camera module 210, e.g. via the transmission component 100, such as via the device connector 106, the device cable 104 and the component coupling part 102.

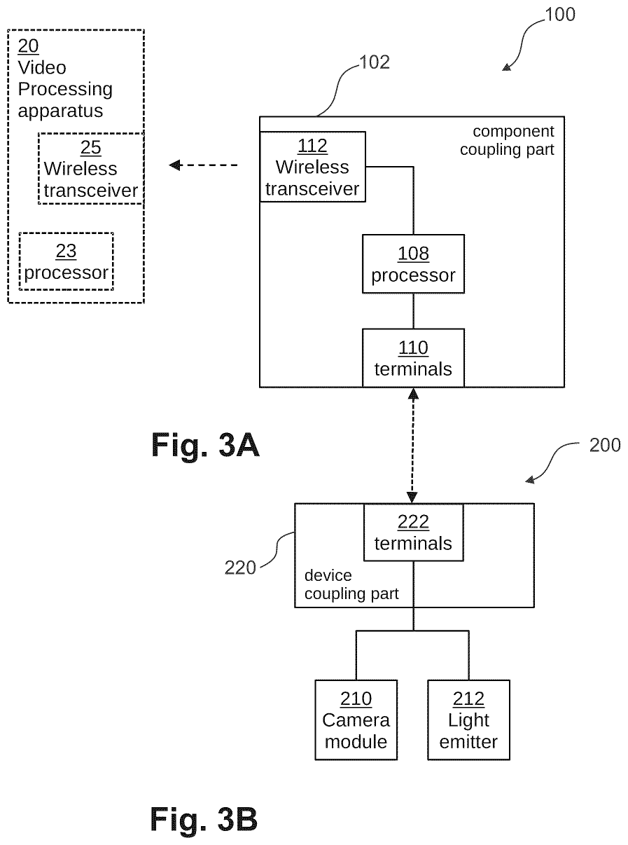

FIG. 3A is a block diagram schematically illustrating another variation of the embodiment of the transmission component 100 of FIG. 1. In the present variation, instead of the device cable 104 and the device connector 106, as illustrated in FIG. 2A, the transmission component 100 comprises a device wireless transceiver 112. FIG. 3B corresponds to FIG. 2B. The device wireless transceiver 112 is adapted to wirelessly communicate with an apparatus wireless transceiver 25 of the video processing apparatus 20, such as the video processing apparatus 20 as illustrated in FIG. 1A. The apparatus wireless transceiver 25 may be an integrated element of the video processing apparatus 20. By integrated element it is mean that the apparatus wireless transceiver 25 is not designed to, and/or capable of, being removed from the video processing apparatus 20 by the user. For example, the apparatus wireless transceiver 25 may be positioned inside the housing 22. However, in other examples, the apparatus wireless transceiver 25 may be connected to the video processing apparatus in a removable manner, in a similar way as the device connector 106, as illustrated in FIG. 1A. The device wireless transceiver 112 is adapted to wirelessly transmit the image data and/or the data representative of the image data to the video processing apparatus 20, e.g. to the apparatus wireless transceiver 25 of the video processing apparatus 20.

In the present disclosure a “transceiver” is meant as being either or both of a transmitter and a receiver. Thus, in one example, the device wireless transceiver 112 may be a transmitter and the apparatus wireless transceiver 25 may be a receiver. In another example, the device wireless transceiver 112 comprises both a transmitter and a receiver and/or the apparatus wireless transceiver 25 comprises both a transmitter and a receiver.

Like above, the transmission component 100 may, also comprise a wireless transceiver 112 and facilitate transmission of data and/or signals from the video processing apparatus to the endoscope 200, such as to the camera module 210 and/or the light emitter 212. For example, camera configuration data may be transmitted from the video processing apparatus to the endoscope 200/camera module 210, e.g. via the transmission component 100, such as via the device wireless transceiver 112. Thus, with a device wireless transceiver 112 that comprises both a transmitter and a receiver, the endoscope 200 may receive configuration data and transmit image data.

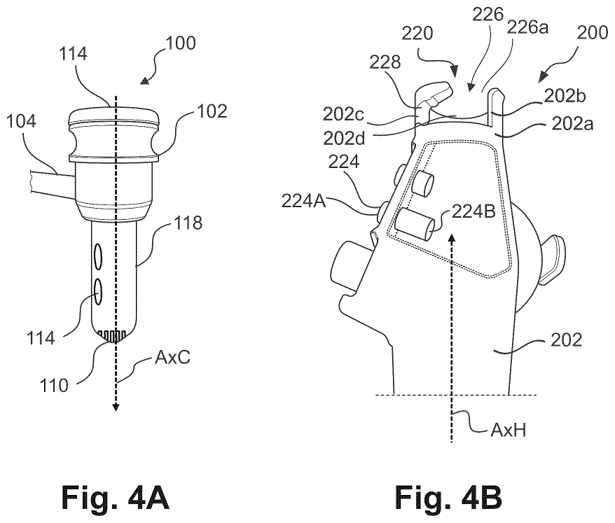

FIGS. 4A and 4B schematically illustrate variations of, respectively, the transmission component 100 and the device coupling part 220 of the endoscope 200. As shown, an outer shell part of the endoscope 200 is removed to show some internal parts of the device coupling part 220.

The component coupling part 102 has a primary coupling element 118. The primary coupling element is elongated and extends along a coupling part axis AxC. The device coupling part 220 has a secondary coupling element 226. The secondary coupling element 226 forms a cavity 226a adapted to receive the primary coupling element 118. The handle 202 is an elongate element extending along the handle axis AxH. The cavity 226a of the secondary coupling element 226 may extend along a handle axis AxH of the handle 202. The handle 202 comprises a wall 202a comprising the cavity 226a and a cut-out 202b on the wall comprising a lateral portion 202c and a central portion 202d. The lateral portion 202c extends from the central portion 202d, which forms a longitudinal slot that extends longitudinally and distally of the lateral portion 202c.

The device coupling part 220 comprises a twist lock 228. The twist lock is adapted to engage by rotation of the handle 202, e.g. around the coupling part axis AxC and/or the handle axis AxH, with a protruding element of the component coupling part 102, e.g. of the primary coupling element 118, when the primary coupling element 118 is fully received by the secondary coupling element 226. As shown, the protruding element engaging with the twist lock may be a part of the device cable 104. The twist lock 228 is formed by the lateral portion 202c and the central portion 202d.

As illustrated in FIG. 4A, the component coupling part 102 may comprise one or more physical activatable input mechanisms 114. Activation of the one or more physical activatable input mechanisms 114 may cause the transmission component 100 to transmit one or more input mechanism signals to the video processing apparatus indicative of activation of the one or more physical activatable input mechanisms 114. Accordingly, a user may activate a function of the video processing apparatus 20 by activation of one of the physical activatable input mechanisms 114. As shown, the one or more physical activatable input mechanisms 114 may be provided on a part received by the device coupling part 220, when the component coupling part 102 is connected with the device coupling part 220. The handle 202 of the endoscope 200, such as the device coupling part 220, comprises one or more communicator elements 224. The one or more communicator elements 224 may be buttons, plungers, levers etc. The one or more communicator elements 224 is adapted to activate the one or more physical activatable input mechanisms 114, when being manipulated by a user. For example, as illustrated, the communicator elements 224 may extend from a first side 224A exteriorly of the handle 202 to a second side 224B interiorly of the handle 202. The second side 224B is adapted to contact a respective physical activatable input mechanism of the one or more physical activatable input mechanisms 114. Thereby, a user may actuate a communicator element 224 on the endoscope 200 to transmit a signal to the video processing apparatus 20. Advantageously, the user can indirectly engage the physical activatable input mechanisms 114 using the communicator elements 224, which the user can reach with fingers of the same hand engaging the actuation mechanism 214. Additionally, the user does not have to contact the transmission component 100, which is reusable, to generate the signal, thus potentially preventing contamination of the transmission component 100.

In some examples, a physical activatable input mechanism 114 may be directly depressible by the user. The physical activatable input mechanism 114 may be positioned on an exposed portion of the component coupling part 102, meaning a portion accessible to the user when the primary coupling element 118 is fully received by the secondary coupling element 226. The primary coupling element 118 may be positioned on a top portion of the component coupling part 102. The physical activatable input mechanisms 114 may be a lever, a switch or similar.

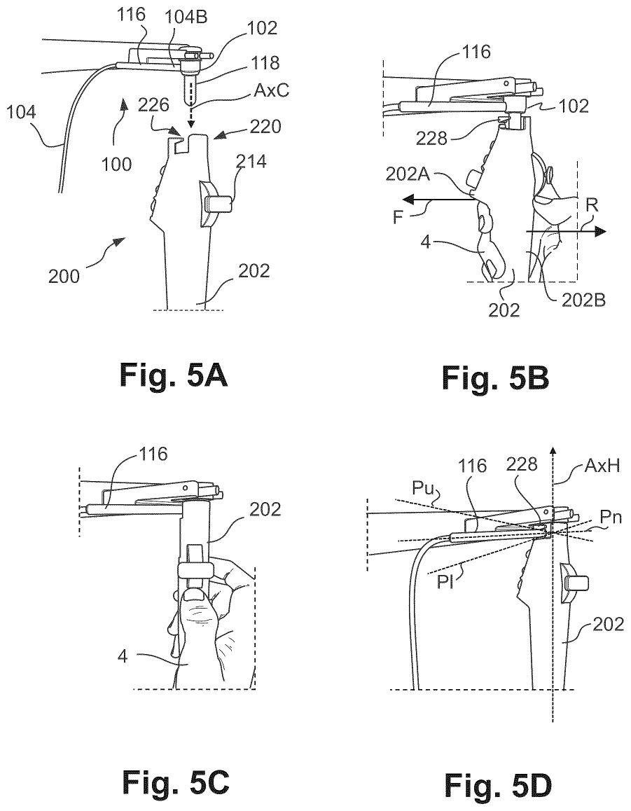

FIGS. 5A-D schematically illustrate a variation of the transmission component 100, the endoscope 200 and a connection between the component coupling part 102 and the device coupling part 220. FIG. 5A is an exploded view, in which the transmission component 100 is not connected to the endoscope 200, and FIGS. 5B-5D are unexploded views, showing how to form the connection between the transmission component 100 and the endoscope 200. As shown, the stiffened portion 116 of the device cable 104 extends at the second cable end 104B. The stiffened portion may be substantially straight. Alternatively, the stiffened portion may be curved either upwards and/or away from the operator. To attach the endoscope 200 to the transmission component 100, the primary coupling element 118 is inserted into the secondary coupling element 226, along the coupling part axis AxC and the handle axis AxH. In other words, the elongate element of the primary coupling element 118 is inserted into the cavity 226a of the secondary coupling element 226.

The twist lock 228 is adapted to engage by rotation of the handle 202, e.g. around the coupling part axis AxC and/or the handle axis AxH, with a protruding element of the component coupling part 102, e.g. of the primary coupling element 118. As shown, the protruding element is the stiffened portion 116 of the device cable 104. As seen in FIGS. 50-5D, the handle 202 is aligned with the stiffened portion 116 aligned with the opening of the twist lock, e.g. the central portion 202d of the cut-out 202b (FIG. 5C), and after the primary coupling element 118 is fully received (along the coupling part axis AxC and/or along the handle axis AxH) by the secondary coupling element 226, the handle is rotated (e.g. clockwise or counterclockwise) to lock the device coupling part 220 and the component coupling part 102 when the stiffened portion 116 is received in the lateral portion 202c of the cut-out 202b. While it is possible to lock the device coupling part 220 and the component coupling part 102 when the cable 104 is received in the lateral portion 202c of the cut-out 202b, stiffening the cable 104 via the stiffened portion 116 promotes a stronger, more stable, lock. The stiffened portion 116 may comprise an overmolding of the cable 104, the provision of a sleeve over the cable 104, or a thicker cover over the wires of the cable 104, for example, with the resulting effect that the stiffened portion 116 is stiffer than the cable portion that is not stiffened. The stiffened portion 116 may be substantially rigid. The stiffened portion may comprise a micro-switch, optical sensor, pressure sensor or any other sensor configured to send a lock signal to the processor 108 indicative of the visualization apparatus being locked with the component coupling part. The lock signal may be used to automatically reel out or reel in a cable (see FIG. 12).

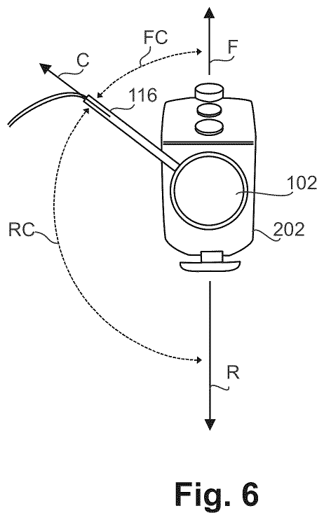

As best seen in FIGS. 5B and 5C, the handle 202 is adapted to be gripped by a first hand 4 of an operator/user such that a back side 202B of the handle 202 is facing in a backwards direction R towards the wrist of the first hand 4 and a front side 202A is facing in a forward direction F opposite the backward direction R. The first hand may be a left hand or a right hand. For illustrative purposes, the figures show a right hand gripping the handle 202, although it may be more customary to grip an endoscope with the left hand. When the component coupling part 102 is connected with the device coupling part 220, as illustrated in FIG. 5D, the stiffened portion 116 may extend in a cable direction between the forward direction F and the backwards direction R, shown in FIG. 6.

As best seen in FIG. 5D, when the component coupling part 102 is connected with the device coupling part 220, the stiffened portion 116 of the device cable 104 may extend in a certain direction relative to the handle 202, such as relative to the handle axis AxH. For example, the stiffened portion 116 of the device cable 104 may extend in a normal plane Pn normal to the handle axis AxH, as illustrated. In some examples, the stiffened portion 116 may extend at an angle relative to the normal plane Pn normal to the handle axis AxH, and/or the stiffened portion 116 may be curved upwards or downwards. In such examples, the stiffened portion 116 may extend between an upper plane Pu and a lower plane PI. The upper plane Pu may form an angle of less than 15 degrees, e.g. +15 degrees, e.g. between 0 and 15 degrees, relative to the normal plane Pn. The lower plane PI may form an angle of less than 15 degrees, e.g. −15 degrees, e.g. between −15 and 0 degrees, relative to the normal plane Pn. The lower plane PI may form an angle of 30 degrees relative to the upper plane Pu. The lower plane PI, the upper plane Pu and the normal plane Pn may intersect the handle axis AxH at the same point.

FIG. 6 schematically illustrates a top view of the arrangement illustrating that the stiffened portion 116 extends in a cable direction C, and that the cable direction C is between the forward direction F and the backwards direction R. The cable direction C may be perpendicular to the coupling part axis AxH (see FIG. 4). An angle FC between the cable direction C and the forward direction F may be less than 90 degrees, such as between 5 and 85 degrees, e.g. 60 degrees or 45 degrees. An angle RC between the cable direction C and the backward direction R may be more than 90 degrees, such as between 95 degrees and 175 degrees, e.g. 120 degrees or 135 degrees.

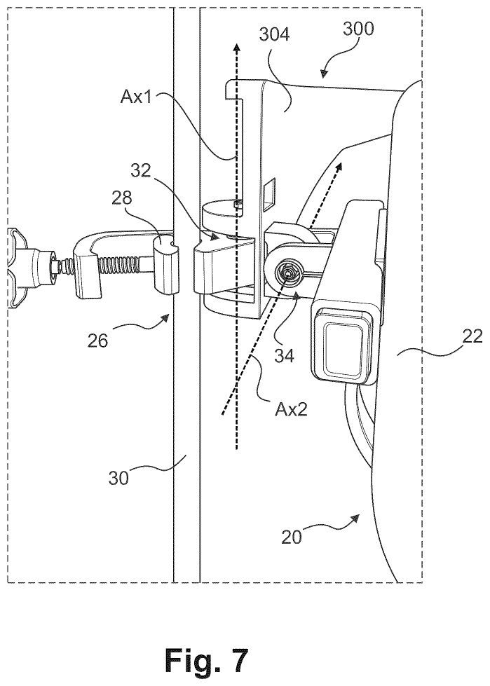

FIG. 7 schematically illustrates an exemplary mounting assembly 26 adapted to attach the housing 22 of the video processing apparatus 20 and the support bracket 300 to a mount 30, e.g. a stand, such as an IV-pole. The video processing apparatus 22 may comprise the mounting assembly 26.

The mounting assembly 26 has a fastener 28, e.g. a clamp, adapted to attach the video processing apparatus 20 to a mount 30. The mounting assembly 26 comprises a first hinge 32 rotatable around a first axis Ax1, e.g. vertically, and a second hinge 34 rotatable around a second axis Ax2, e.g. horizontally. The first axis Ax1 and the second axis Ax2 may be perpendicular. The first hinge 32 is arranged between the fastener 28 and the second hinge 34. The second hinge 34 is arranged between the first hinge 32 and the housing 22 of the video processing apparatus 20. The proximal support bracket part 304 is coupled to the mounting assembly 26 between the first hinge 32 and the second hinge 34. Thereby, the support bracket 300 follows the rotation of the first hinge 32, i.e. around the first axis Ax1, but does not follow the rotation of the second hinge 34, i.e. around the second axis Ax1. Thus, tilting the housing 22 of the video processing apparatus 20 will not affect the orientation of the support bracket 300. On the other hand, turning the housing 22 affects the orientation of the support bracket 300 correspondingly.

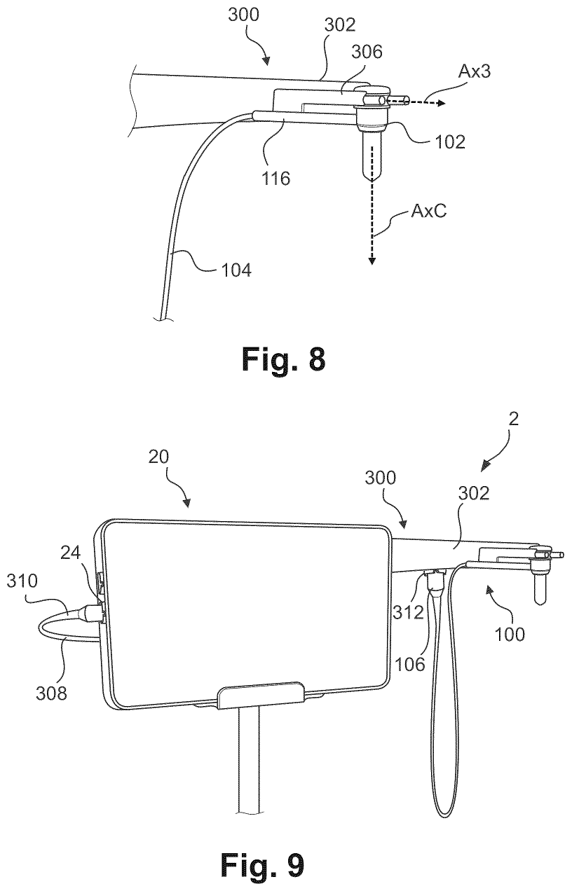

FIG. 8 schematically illustrates an example of the support bracket 300 and the component coupling part 102 retained by the support bracket 300. As seen, the distal support bracket part 302 includes the coupling part retainer 306, which is adapted to retain the component coupling part 102.

The coupling part retainer 306 is adapted to receive and/or supply the component coupling part 102 by linear movement of the component coupling part 102 along a third axis Ax3. Similarly, the component coupling part 102 is adapted to engage with the coupling part retainer 306 by the linear movement along the third axis Ax3. The coupling part axis AxC may be non-parallel with the third axis Ax3, when the component coupling part 102 is received by the coupling part retainer 306. For example, the coupling part axis AxC and the third axis Ax3 may form an angle between 45-90 degrees. For example, the coupling part axis AxC and the third axis Ax3 may be perpendicular.

The coupling part retainer 306 and/or the component coupling part 102 may be adapted to prevent rotation of the component coupling part 102 relative to the coupling part retainer 306, when the component coupling part 102 is received by the coupling part retainer 306. To prevent rotation, the part of the component coupling part 102 engaging with the coupling part retainer 306 may have a non-circular shape, e.g. an oval shape or an elliptical shape.

The coupling part retainer 306 and/or the component coupling part 102 may be adapted to prevent movement, such as linear movement, of the component coupling part 102 along the coupling part axis AxC and/or along the first axis Ax1 and/or the second axis Ax2 (see FIG. 7), when the component coupling part 102 is received by the coupling part retainer 306. To prevent movement, such as linear movement, of the component coupling part 102 along the coupling part axis AxC (and/or along the first axis Ax1 and/or the second axis Ax2), the part of the component coupling part 102 engaging with the coupling part retainer 306 may have a concave shape.

In the present example, the user may couple the handle 202 of the endoscope 200 to the component coupling part 102, as explained in relation to FIGS. 5A-5D, and subsequently remove the component coupling part 102 (while being attached to the handle 202) from the coupling part retainer 306 by moving the handle linearly away from the coupling part retainer 306 along the third axis Ax3 (FIG. 8). Similarly, e.g. after use, the user may return the component coupling part 102 (while being attached to the handle 202) to the coupling part retainer 306 by aligning the component coupling part 102 and the coupling part retainer 306, and position the component coupling part 102 in the coupling part retainer 306 by movement along the third axis Ax3. The user may let both the component coupling part 102 and the attached endoscope 200 be held by the coupling part retainer 306, as seen in FIG. 5D. To decouple the endoscope 200 from the component coupling part 102, the user may turn the handle 202, as illustrated in FIG. 5C, and remove the handle 202 and the endoscope 200 from the component coupling part 102 along the coupling part axis AxC. Thus, by the present disclosure, both coupling and decoupling of the endoscope 200 and component coupling part 102 may be performed without a need to touch the component coupling part 102 or the transmission component 100, which may be reusable. Thereby the risk of cross contamination may be reduced.

FIG. 9 schematically illustrates a medical visualization system 2 like the medical visualization system 2 of FIG. 1A. However, in FIG. 9, the support bracket 300 comprises a support cable 308. The support cable 308 extends (e.g. inside the support bracket) between the distal support bracket part 302 and the proximal support bracket part 304. A first end 310 of the support cable 308 is arranged at and/or extends from the proximal support bracket part 304, and is adapted to connect to the video processing apparatus 20, as illustrated. A second end 312 of the support cable 308 is arranged at the distal support bracket part 302 and is adapted to connect to the transmission component 100. For example, as illustrated, the second end 312 of the support cable 308 may terminate in a connection port similar to the connection port 24 of the video processing apparatus 20, and the device connector 106 of the transmission component 100 may be connected with the connection port formed by the second end 312 of the support cable 308. The support cable 308 may be reusable and may simplify and/or facilitate connection of the transmission component 100 by the operator.

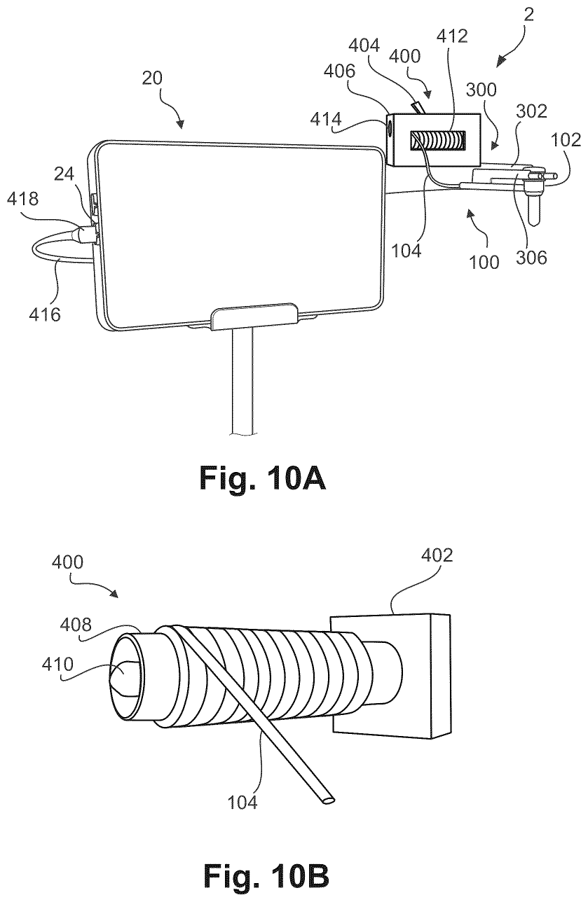

FIG. 10A schematically illustrates an example of a medical visualization system 2 like the medical visualization system 2 of FIG. 1A or FIG. 9. However, in FIG. 10A, the medical visualization system 2 comprises a cable reel 400. Internal parts of the cable reel 400 are schematically illustrated in FIG. 10B. The cable reel 400 may comprise a cable reel housing 406, which is not shown in FIG. 10B to allow visibility of the internal parts of the cable reel 400. FIGS. 10A and 10B are described together in the following. The cable reel 400 is adapted to reel in at least a portion of the device cable 104. The cable reel housing 406 may enclose the portion of the device cable 104 when reeled in. In some variations, the cable reel 400 forms part of the support bracket 300. In other variations, the cable reel 400 forms part of the transmission component 100. A connector cable 416 may extend from the cable reel 400 and may be adapted to connect to the video processing apparatus 20 at its first end 418. The connector cable 416 may be adapted to connect the video processing apparatus 20 and the device cable 104. In some examples, the connector cable 416 is the support cable 308 as previously described. In other examples, the connector cable 416 may form part of the cable reel 400. In further examples, the connector cable 416 is a separate cable adapted to connect the cable reel 400 to the video processing apparatus 20.

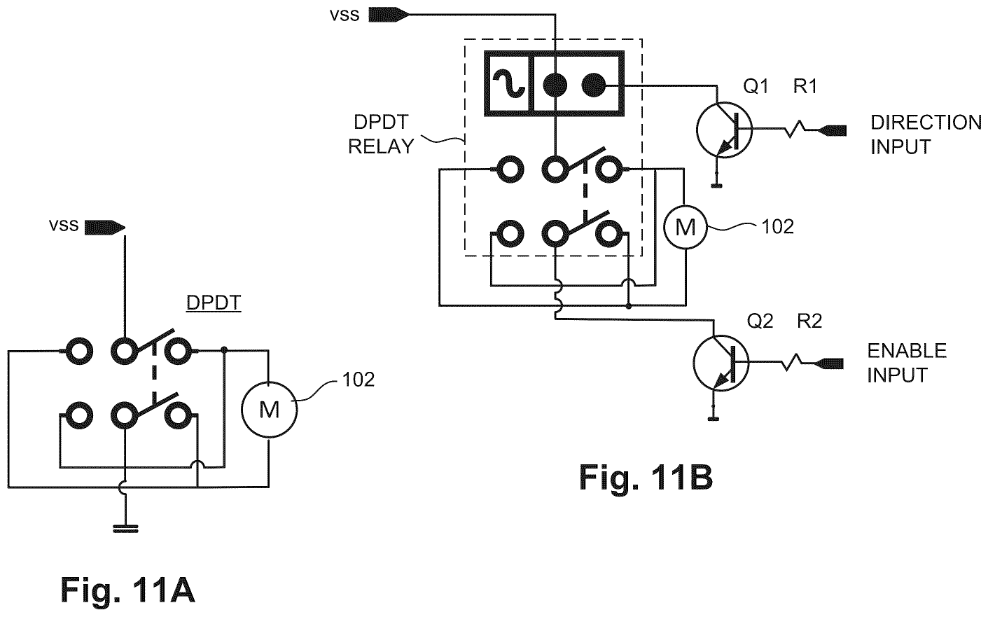

The cable reel 400 may be driven by an actuator 402. The actuator 402 may be a rotary actuator, e.g. an electrical motor, such as a DC motor. For example, the actuator 402 may drive the cable reel 400 such that the device cable 104 is reeled in and out as appropriate. For example, this may be advantageous so as to reduce any influence of the cable reel on the operator's ability to control the endoscope being attached to the device cable 104, e.g. by the component coupling part 102. The actuator may be controlled based on user inputs, e.g. via the video processing apparatus 20 (e.g. via touch screen input on the video processing apparatus), or via a dedicated input device(s) 404 (e.g. a switch, button, lever or similar) of the cable reel 400. For example, a first position of the input device 404 may cause the cable reel 400 to reel in the device cable 104, and a second position of the input device 404 may cause the cable reel 400 to unreel the device cable 104. A double-pole double-throw (DPDT) switch, for example, has on-off-on positions, the middle position being neutral, where the first and third positions can be wired to provide opposite polarities to the motor (M). An example of a DPDT switch is depicted in FIG. 11A, where VSS denotes power/voltage supply source, such as a battery or a voltage wire from the video processing apparatus. The DPDT switch can be substituted with two single-pole switches or two buttons, one for forward and the other for reverse rotation. The actuator 402 may be activated to reel in the device cable 104 based on a first control input and/or may be activated to unreel the device cable 104 based on a second control input. The first and/or second control input may be an input from the input device 404 and/or from the video processing apparatus 20. In some examples, the input device 404 may be employed in the coupling part retainer 306. For example, the input device 404 may be implemented as a switch, e.g. a physical switch or a hall switch, detecting the presence of the component coupling part 102 being received in the coupling part retainer 306. Thereby, removal of the component coupling part 102 from the coupling part retainer 306 may cause the cable reel 400, e.g. the actuator 402 of the cable reel 400, to unreel the device cable 104. Alternatively or additionally receipt of the component coupling part 102 in the coupling part retainer 306 may cause the cable reel 400, e.g. the actuator 402 of the cable reel 400, to reel in the device cable 104.

The cable reel comprises a spool 408. The spool 408 is accommodated in the cable reel housing 406. The portion of the device cable 104 is rolled onto the spool 408 when reeled in by the cable reel 400. The spool 408 may be transparent to UV light. For example, the spool 408 may be made from Polymethylmethacrylate (PMMA).

The cable reel 400 may comprise a UV light emitter 410. The UV light emitter 410 is accommodated in the cable reel housing 406. In the illustrated example, the UV light emitter 410 is arranged inside the spool 408. Thereby, in combination with the spool 408 being transparent, the UV light emitter 410 is able to subject the part of the device cable 104 rolled onto the spool 408 to UV light. Alternatively or additionally, the device cable 104 may be transparent and/or partly transparent to UV light. For example, the device cable 104 may comprise a transparent material, e.g. polymer, enclosing the electrical conducing wires. Making the device cable 104 transparent to UV light reduces the risk of parts of the device cable 104 blocking other parts from exposure of the UV light, e.g. if the device cable 104 is coiled up over itself. Thereby, more efficient disinfection of more of the device cable 104 may be achieved.

To contain the UV light within the cable reel housing 406, the opening 412 through which the device cable 104 protrude may be made small or may be covered with bristles or similar to avoid or at least reduce UV light from escaping the cable reel housing 406. The cable reel housing 406 may be opaque to UV light. The UV light emitter 410 may be activated automatically, e.g. after the device cable 104 is rolled onto the spool 408. Alternatively or additionally, the UV light emitter 410 may be activated by a user input, e.g. via the video processing apparatus 20 (e.g. via touch screen input on the video processing apparatus), or via a dedicated second input device 414 (e.g. a switch, button, lever or similar) of the cable reel 400. It may also be envisioned that the opening 412 may be closable, e.g. the opening 412 may comprise a shutter for closing the opening 412. In such an example, activation of the UV light emitter 410 may be activated based on whether the opening 412 is closed. For example, the UV light emitter 410 may be only activated if the opening 412 is closed and/or the UV light emitter 410 may be activated automatically when the opening 412 is closed.

In some examples, the transmission component 100 comprises a counter, e.g. a digital counter 420. The counter may be indicative of the number of times the transmission component 100 has been used and/or may be indicative of the number of times the transmission component 100 (such as the device cable 104) has been subjected to UV light by the UV light emitter 410 and/or may be indicative of deterioration of the transmission component 100. The counter may be an electronically readable counter, e.g. a flash memory or other suitable electronic memory known in the art. The electronically readable counter may be readable by the video processing apparatus 20 which may then indicate to the user the value of the counter, and/or indicate to the user whether it is time to replace the transmission component 100. Alternatively or additionally, the counter may be employed by providing the transmission component with a physical wear sensor 422 to detect a change in the transmission component, e.g. a part changing color over time and/or based on UV light exposure. The counter may be indicative of deterioration of the transmission component 100, such as of the device cable 104 of the transmission component 100, which may be especially relevant when using UV light for disinfection, because UV light can be harsh on materials used for the transmission component 100 such as materials used for the device cable 104. The counter may count activation of the input device 404.

A DPDT relay may also be used instead of a DPDT switch. An example of a DPDT relay is depicted in FIG. 11B. Its function is similar to the DPDT switch except the switch is operated by a coil that is controlled by a direction input. As shown, a transistor Q1 and resistor R1 protect the direction input from the coil. A transistor Q2 and resistor R2 protect an enable input from the coil. The enable input energizes the motor and the direction input determines whether to rotate forward or reverse. These inputs can also be connected directly to the relay if the source of the inputs, e.g. a processor, is capable of direct connection to VSS. A person of ordinary skill in the art will understand how to provide equivalent electronic circuits with different protection circuits, for example, tailored to the chosen processor technology or for other reasons, such as cost. FIG. 12 shows the circuit of FIG. 11B connected to the processor 108. FIG. 12 also shows the UV light connected via a transistor Q3 and a resistor R3 to a sanitation input. A switch is also shown, pulled-up by a resistor R4, so that when the switch is thrown the twist lock input is negative and when the switch is not thrown the input is positive. The advantage of using a processor, whether it is processor 108 or the processor of the video processing apparatus, is that the processor will, via the twist lock input, know when the endoscope is properly connected, thus the processor can determine whether to reel or unreel the device cable 104, and whether and when to sanitize it, thus automating the process. Additionally, the processor can advance the counter 420 each time the device cable 104 is sanitized and generate a warning if the number of sanitazions exceeds a safety threshold. Additionally, a wear sensor 422 is shown. The wear sensor 422 may be an optical sensor configured to detect a color change of a portion of the device cable 104 being repeatedly sanitized. The portion may be treated or provided as a coating or sleeve that it changes color after repeated sanitation via UV treatment. Of course, individual switches or buttons can be connected to the processor as inputs if it is desired to give the operator manual control. Another advantage is that when the processor is in the video processing apparatus, the processor is not discarded with the device cable 104 and the software can be updated, if needed or desired, via a software update to the video processing apparatus rather than separately.

The disclosure has been described with reference to a preferred embodiment. However, the scope of the invention is not limited to the illustrated embodiment, and alterations and modifications can be carried out without deviating from the scope of the invention.

Throughout the description, the use of the terms “first”, “second”, “third”, “fourth”, “primary”, “secondary”, “tertiary” etc. does not imply any particular order or importance but are included to identify individual elements. Furthermore, the labelling of a first element does not imply the presence of a second element and vice versa.

The following items are further variations and examples of the embodiments described with reference to the figures.

1. A medical visualization system comprising a video processing apparatus, an endoscope and a transmission component, the endoscope comprising a camera module adapted to generate image data indicative of a view from the endoscope, a handle, and a device coupling part, the video processing apparatus comprising a housing and a processor adapted to receive the image data and/or data representative of the image data and cause a display to display a live representation of the image data, the transmission component being adapted to transmit the image data and/or the data representative of the image data to the video processing apparatus, the transmission component comprising a component coupling part adapted to couple with the device coupling part.

2. Medical visualization system according to item 1, wherein the transmission component comprises a component processor adapted to receive the image data from the camera module and preprocess the image data to provide the data representative of the image data.

3. Medical visualization system according to item 2, wherein the component coupling part comprises the component processor.

4. Medical visualization system according to any one of the preceding items, wherein the component coupling part comprises one or more physical activatable input mechanisms, and wherein activation of the one or more physical activatable input mechanisms causes the transmission component to transmit one or more input mechanism signals to the video processing apparatus indicative of activation of the one or more physical activatable input mechanisms.

5. Medical visualization system according to item 4, wherein the handle of the endoscope comprises one or more communicator elements adapted to activate the one or more physical activatable input mechanisms upon being manipulated by a user.

6. Medical visualization system according to item 5, wherein a first communicator element of the one or more communicator elements extends from a first side to a second side, wherein the first side is arranged exteriorly of the handle and the second side is arranged interiorly of the handle, and wherein the second side is adapted to contact a first physical activatable input mechanism of the one or more physical activatable input mechanism when the component coupling part is coupled with the device coupling part.

7. Medical visualization system according to any one of the preceding items, wherein the transmission component comprises a device cable extending from a first cable end to a second cable end, wherein the component coupling part is provided at the second cable end.

8. Medical visualization system according to item 7, wherein the device cable and/or the component coupling part is coated with silver.

9. Medical visualization system according to any one of items 7-8, wherein the device cable comprises a stiffened portion extending from the second cable end towards the first cable end, and wherein the stiffened portion has a length of between 20-150 mm from the second cable end.

10. Medical visualization system according to item 9, wherein the stiffened portion is substantially straight.

11. Medical visualization system according to any one of items 9-10, wherein the handle is an elongate element extending along a handle axis and wherein the stiffened portion of the device cable extends in a plane normal to the handle axis.

12. Medical visualization system according to any one of items 9-11, wherein the handle is adapted to be gripped by a first hand of an operator such that a back side of the handle is facing in a backwards direction towards the wrist of the first hand and a front side is facing in a forward direction opposite the backward direction, and wherein the stiffened portion extends in a cable direction between the forward direction and the backwards direction, wherein an angle between the cable direction and the forward direction is between 5 and 85 degrees.

13. Medical visualization system according to any one of items 7-12 comprising a cable reel adapted to reel in at least a portion of the device cable.

14. Medical visualization system according to item 13, wherein the cable reel is driven by an actuator, and wherein the actuator is activated to reel in the portion of the device cable based on a first control input.

15. Medical visualization system according to any one of items 13-14, wherein the cable reel comprises a cable reel housing enclosing the portion of the device cable when reeled in.

16. Medical visualization system according to item 15, wherein the cable reel comprises a spool accommodated in the cable reel housing and wherein the portion of the device cable is rolled onto the spool when reeled in by the cable reel, preferably the spool is transparent to UV light.

17. Medical visualization system according to any one of items 15-16, wherein the cable reel comprises an UV light emitter accommodated in the cable reel housing.

18. Medical visualization system according to any one of the preceding items, wherein the transmission component comprises a counter indicative of deterioration of the transmission component.

19. Medical visualization system according to any one of the preceding items, wherein the transmission component comprises a device wireless transceiver adapted to wirelessly communicate with an apparatus wireless transceiver of the video processing apparatus, the device wireless transceiver being adapted to wirelessly transmit the image data and/or the data representative of the image data to the apparatus wireless transceiver.

20. Medical visualization system according to any one of the preceding items, wherein the component coupling part has a primary coupling element being elongated and extending along a coupling part axis, and wherein the device coupling part has a secondary coupling element forming a cavity adapted to receive the primary coupling element.

21. Medical visualization system according to item 20, wherein the device coupling part comprises a twist lock adapted to engage by rotation of the handle with a protruding element of the component coupling part when the primary coupling element is fully received by the secondary coupling element.

22. Medical visualization system according to any one of the preceding items, wherein the device coupling part comprises one or more device terminals electronically connected to the camera module, and the component coupling part comprises one or more component terminals adapted to connect to the one or more device terminals of the device coupling part when the component coupling part is coupled with the device coupling part.

23. Medical visualization system according to any one of the preceding items further comprising a support bracket having a distal support bracket part and a proximal support bracket part, and wherein the distal support bracket part includes a coupling part retainer adapted to retain the component coupling part of the transmission component.

24. Medical visualization system according to item 23, wherein the proximal support bracket part is adapted to be coupled to the housing of the video processing apparatus, e.g. such that the support bracket follows rotation of the housing around a first axis and do not follow rotation of the housing around a second axis perpendicular to the first axis.

25. Medical visualization system according to item 24, wherein the video processing apparatus comprises a mounting assembly having a fastener adapted to attach the video processing apparatus to a mount, a first hinge rotatable around a first axis, and a second hinge rotatable around a second axis, wherein the first hinge is arranged between the fastener and the second hinge and the second hinge is arranged between the first hinge and the housing of the video processing apparatus, and wherein the proximal support bracket part is coupled to the mounting assembly between the first hinge and the second hinge.

26. Medical visualization system according to any one of items 23-25, wherein the coupling part retainer is adapted to receive the component coupling part by linear movement of the component coupling part along a third axis, and/or wherein the component coupling part is adapted to engage with the coupling part retainer by linear movement of the component coupling part along the third axis.

27. Medical visualization system according to item 26 as dependent on at least item 20, wherein the coupling part axis is non-parallel with the third axis, such as between 45-90 degrees angled to the third axis, such as perpendicular to the third axis, when the component coupling part is received by the coupling part retainer.

28. Medical visualization system according to any one of items 23-27, wherein the coupling part retainer is adapted to prevent rotation of the component coupling part when received by the coupling part retainer, and/or wherein the component coupling part is adapted to prevent rotation of the component coupling part when received by the coupling part retainer.

29. Medical visualization system according to any one of items 23-28, wherein the coupling part retainer is adapted to prevent movement along the first axis and/or second axis of the component coupling part when received by the coupling part retainer, and/or wherein the component coupling part is adapted to prevent movement along the first axis and/or second axis of the component coupling part when received by the coupling part retainer.

30. Medical visualization system according to any one of items 23-29, wherein the support bracket comprises a support cable extending between the distal support bracket part and the proximal support bracket part, a first end of the support cable is arranged at the proximal support bracket part and is adapted to connect to the video processing apparatus, and a second end of the support cable is arranged at the distal support bracket part and is adapted to connect to the transmission component.

31. Medical visualization system according to any one of items 23-30 as dependent on at least item 13, wherein the support bracket comprises the cable reel.

32. Medical visualization system according to any one of items 23-31 as dependent on at least item 14, wherein the first control input is triggered by the component coupling part being received by the coupling part retainer, e.g. the coupling part retainer comprises a switch subjective to receipt of the component coupling part and the switch triggers the first control input.

33. Medical visualization system according to any one of the preceding items, wherein the endoscope comprises an insertion cord extending from the handle to a distal cord portion, and wherein the view from the endoscope is a view from the distal cord portion of the insertion cord, and wherein the handle comprises a control mechanism adapted to receive an input in a first input direction, and wherein the input in the first input direction causes a bendable section of the insertion cord to bend in a first bending direction.

34. Medical visualization system according to any one of the preceding items, wherein the endoscope is disposable, and the transmission component is reusable and adapted to be coupled to a plurality of endoscopes.

35. Medical visualization system according to any one of the preceding items, wherein the video processing apparatus comprises the display, preferably the display is accommodated in the housing of the video processing apparatus.

36. A transmission component for a medical visualization system further comprising a video processing apparatus, and an endoscope, the endoscope comprising a camera module adapted to generate image data indicative of a view from the endoscope, a handle, and a device coupling part, the video processing apparatus comprising a housing and a processor adapted to receive the image data and/or data representative of the image data and cause a display to display a live representation of the image data, the transmission component being adapted to transmit the image data and/or the data representative of the image data to the video processing apparatus, the transmission component comprising a component coupling part adapted to couple with the device coupling part.

37. Transmission component according to item 36, wherein the transmission component comprises a component processor adapted to receive the image data from the camera module and preprocess the image data to provide the data representative of the image data.

38. Transmission component according to item 37, wherein the component coupling part comprises the component processor.

39. Transmission component according to any one of items 36-38, wherein the component coupling part comprises one or more physical activatable input mechanisms, and wherein activation of the one or more physical activatable input mechanisms causes the transmission component to transmit one or more input mechanism signals to the video processing apparatus indicative of activation of the one or more physical activatable input mechanisms.

40. Transmission component according to item 39, wherein the one or more physical activatable input mechanisms are adapted to be activated by one or more communicator elements of the handle of the endoscope.

41. Transmission component according to any one of items 36-40, wherein the transmission component comprises a device cable extending from a first cable end to a second cable end, wherein the component coupling part is provided at the second cable end.

42. Transmission component according to item 41, wherein the device cable and/or the component coupling part is coated with silver.

43. Transmission component according to any one of items 41-42, wherein the device cable comprises a stiffened portion extending from the second cable end towards the first cable end, and wherein the stiffened portion has a length of between 20-150 mm from the second cable end.

44. Transmission component according to item 43, wherein the stiffened portion is substantially straight.

45. Transmission component according to any one of items 43-44, wherein the stiffened portion extends in a plane normal to a handle axis when the component coupling part is coupled with the device coupling part, wherein the handle is an elongate element extending along the handle axis.

46. Transmission component according to any one of items 43-45, wherein the stiffened portion extends in a cable direction between a forward direction and a backwards direction when the component coupling part is coupled with the device coupling part, wherein the backwards direction is towards the wrist of a first hand of an operator gripping the handle and the forward direction is opposite the backwards direction, and wherein an angle between the cable direction and the forward direction is between 5 and 85 degrees.

47. Transmission component according to any one of items 41-46 comprising a cable reel adapted to reel in at least a portion of the device cable.

48. Transmission component according to item 47, wherein the cable reel is driven by an actuator, and wherein the actuator is activated to reel in the portion of the device cable based on a first control input.

49. Transmission component according to any one of items 47-48, wherein the cable reel comprises a cable reel housing enclosing the portion of the device cable when reeled in.

50. Transmission component according to item 49, wherein the cable reel comprises a spool accommodated in the cable reel housing and wherein the portion of the device cable is rolled onto the spool when reeled in by the cable reel, preferably the spool is transparent to UV light.

51. Transmission component according to any one of items 49-50, wherein the cable reel comprises an UV light emitter accommodated in the cable reel housing.

52. Transmission component according to any one of items 36-51, wherein the transmission component comprises a counter indicative of deterioration of the transmission component.

53. Transmission component according to any one of items 36-52, wherein the transmission component comprises a device wireless transceiver adapted to wirelessly communicate with an apparatus wireless transceiver of the video processing apparatus, the device wireless transceiver being adapted to wirelessly transmit the image data and/or the data representative of the image data to the apparatus wireless transceiver.

54. Transmission component according to any one of items 36-53, wherein the component coupling part has a primary coupling element being elongated and extending along a coupling part axis and being adapted to be received by a secondary coupling element of the device coupling part forming a cavity.

55. Transmission component according to any one of items 36-54, wherein the component coupling part comprises one or more component terminals adapted to connect to one or more device terminals of the device coupling part when the component coupling part is coupled with the device coupling part.

56. An endoscope for a medical visualization system further comprising a video processing apparatus and a transmission component, the endoscope comprising a camera module adapted to generate image data indicative of a view from the endoscope, a handle, and a device coupling part, the video processing apparatus comprising a housing and a processor adapted to receive the image data and/or data representative of the image data and cause a display to display a live representation of the image data, the transmission component being adapted to transmit the image data and/or the data representative of the image data to the video processing apparatus, the transmission component comprising a component coupling part adapted to couple with the device coupling part.

57. Endoscope according to item 56, wherein the handle of the endoscope comprises one or more communicator elements adapted to, upon being manipulated by a user, activate one or more physical activatable input mechanisms of the component coupling part when the component coupling part is coupled with the device coupling part.

58. Endoscope according to item 57, wherein a first communicator element of the one or more communicator elements extends from a first side to a second side, wherein the first side is arranged exteriorly of the handle and the second side is arranged interiorly of the handle, and wherein the second side is adapted to contact a first physical activatable input mechanism of the one or more physical activatable input mechanism when the component coupling part is coupled with the device coupling part.

59. Endoscope according to any one of items 56-58, wherein the device coupling part has a secondary coupling element forming a cavity adapted to receive a primary coupling element of the component coupling part extending along a coupling part axis.

60. Endoscope according to item 59, wherein the device coupling part comprises a twist lock adapted to engage by rotation of the handle with a protruding element of the component coupling part when the primary coupling element is fully received by the secondary coupling element.

61. Endoscope according to any one of items 56-60, wherein the device coupling part comprises one or more device terminals electronically connected to the camera module and adapted to connect to one or more component terminals of the component coupling part when the component coupling part is coupled with the device coupling part.

62. Endoscope according to any one of items 56-61, wherein the endoscope comprises an insertion cord extending from the handle to a distal cord portion, and wherein the view from the endoscope is a view from the distal cord portion of the insertion cord, and wherein the handle comprises a control mechanism adapted to receive an input in a first input direction, and wherein the input in the first input direction causes a bendable section of the insertion cord to bend in a first bending direction.

63. Endoscope according to any one of items 56-62, wherein the endoscope is disposable, and the transmission component is reusable and adapted to be coupled to a plurality of endoscopes.

64. A cable reel for a medical visualization system further comprising a video processing apparatus, an endoscope and a transmission component, the endoscope comprising a camera module adapted to generate image data indicative of a view from the endoscope, a handle, and a device coupling part, the video processing apparatus comprising a housing and a processor adapted to receive the image data and/or data representative of the image data and cause a display to display a live representation of the image data, the transmission component being adapted to transmit the image data and/or the data representative of the image data to the video processing apparatus, the transmission component comprising a device cable extending from a first cable end to a second cable end, and wherein a component coupling part is provided at the second cable end, the component coupling part being adapted to couple with the device coupling part, the cable reel being adapted to reel in at least a portion of the device cable.

65. Cable reel according to item 64, wherein the cable reel is driven by an actuator, and wherein the actuator is activated to reel in the portion of the device cable based on a first control input.

66. Cable reel according to any one of items 64-65, wherein the cable reel comprises a cable reel housing enclosing the portion of the device cable when reeled in.

67. Cable reel according to item 66, wherein the cable reel comprises a spool accommodated in the cable reel housing and wherein the portion of the device cable is rolled onto the spool when reeled in by the cable reel, preferably the spool is transparent to UV light.

68. Cable reel according to any one of items 66-67, wherein the cable reel comprises a UV light emitter accommodated in the cable reel housing.

69. A support bracket for a medical visualization system further comprising a video processing apparatus, an endoscope and a transmission component, the endoscope comprising a camera module adapted to generate image data indicative of a view from the endoscope, a handle, and a device coupling part, the video processing apparatus comprising a housing and a processor adapted to receive the image data and/or data representative of the image data and cause a display to display a live representation of the image data, the transmission component being adapted to transmit the image data and/or the data representative of the image data to the video processing apparatus, the transmission component comprising a component coupling part adapted to couple with the device coupling part, the support bracket has a distal support bracket part and a proximal support bracket part, and wherein the distal support bracket part includes a coupling part retainer adapted to retain the component coupling part of the transmission component.

70. Support bracket according to item 69, wherein the proximal support bracket part is adapted to be coupled to the housing of the video processing apparatus, e.g. such that the support bracket follows rotation of the housing around a first axis and do not follow rotation of the housing around a second axis perpendicular to the first axis.

71. Support bracket according to item 70, wherein the proximal support bracket part is adapted to be coupled to a mounting assembly of the video processing apparatus between a first hinge and a second hinge of the mounting assembly.

72. Support bracket according to any one of items 69-71, wherein the coupling part retainer is adapted to receive the component coupling part by linear movement of the component coupling part along a third axis.

73. Support bracket according to any one of items 69-72, wherein the coupling part retainer is adapted to prevent rotation of the component coupling part when received by the coupling part retainer.

74. Support bracket according to any one of items 69-73, wherein the coupling part retainer is adapted to prevent movement along a first axis and/or a second axis of the component coupling part when received by the coupling part retainer.

75. Support bracket according to any one of items 69-74, wherein the support bracket comprises a support cable extending between the distal support bracket part and the proximal support bracket part, a first end of the support cable is arranged at the proximal support bracket part and is adapted to connect to the video processing apparatus, and a second end of the support cable is arranged at the distal support bracket part and is adapted to connect to the transmission component.

76. Support bracket according to any one of items 69-75, wherein the support bracket comprises a cable reel adapted to reel in at least a portion of a device cable of the transmission component.

77. Support bracket according to item 76, wherein the cable reel is driven by an actuator, and wherein the actuator is activated to reel in the portion of the device cable based on a first control input.

78. Support bracket according to item 77, wherein the first control input is triggered by the component coupling part being received by the coupling part retainer, e.g. the coupling part retainer comprises a switch subjective to receipt of the component coupling part and the switch triggers the first control input.

### LIST OF REFERENCES

2 medical visualization system
4 handle
20 video processing apparatus
21 display
22 housing
23 processor
24 connection port(s)
25 apparatus wireless transceiver
26 mounting assembly
28 fastener
30 mount
32 first hinge
34 second hinge
100 transmission component
102 component coupling part
104 device cable
104A, 104B ends of device cable
106 device connector
108 component processor
110 component terminal(s)
112 device wireless transceiver
114 input mechanisms
116 stiffened portion
118 primary coupling element
200 endoscope
202 handle
202A, 202B sides of handle
203 insertion cord
204 insertion tube
206 bending section
208 tip/distal cord portion
210 camera module
212 light emitter
214 steering/control mechanism
220 device coupling part
222 device terminal(s)
224 communicator elements
224B, 224B ends of communicator element(s)
226 secondary coupling element
228 twist lock
300 support bracket
302 distal support bracket part
304 proximal support bracket part
306 coupling part retainer
308 support cable
310 first end of support cable
312 second end of support cable
400 cable reel
402 actuator
404 input device
406 cable reel housing
408 spool
410 UV light emitter
412 opening
414 second input device
416 connector cable
418 first end of connector cable
Ax1 first axis
Ax2 second axis
Ax3 third axis
AxH handle axis
AxC coupling part axis
R backward direction
F forward direction
C cable direction
RC angle between cable and backward directions
FC angle between cable and forward directions
Pn normal plane
Pu upper plane
PI lower plane

1. A medical visualization system comprising a video processing apparatus, an endoscope and a transmission component,
the endoscope comprising a camera module adapted to generate image data indicative of a view from the endoscope, a handle, and a device coupling part,
the video processing apparatus comprising a housing and a processor adapted to receive the image data and/or data representative of the image data and cause a display to display a live representation of the image data,
the transmission component comprising a component coupling part adapted to couple with the device coupling part and including a component processor adapted to receive the image data from the camera module and preprocess the image data to provide data representative of the image data, the transmission component being adapted to transmit the data representative of the image data to the video processing apparatus.

2. The medical visualization system of claim 1, wherein the component coupling part comprises one or more physical activatable input mechanisms, wherein activation of the one or more physical activatable input mechanisms causes the transmission component to transmit one or more input mechanism signals to the video processing apparatus indicative of activation of the one or more physical activatable input mechanisms, wherein the handle of the endoscope comprises one or more communicator elements adapted to activate the one or more physical activatable input mechanisms upon being manipulated by a user, wherein a first communicator element of the one or more communicator elements extends from a first side to a second side of a wall of the handle, wherein the first side is arranged exteriorly of the handle and the second side is arranged interiorly of the handle, and wherein the second side is adapted to contact a first physical activatable input mechanism of the one or more physical activatable input mechanism when the component coupling part is coupled with the device coupling part.

3. The medical visualization system of claim 1, wherein the component coupling part comprises one or more physical activatable input mechanisms, and wherein activation of the one or more physical activatable input mechanisms causes the transmission component to transmit one or more input mechanism signals to the video processing apparatus indicative of activation of the one or more physical activatable input mechanisms.

4. The medical visualization system of claim 3, wherein the handle of the endoscope comprises one or more communicator elements adapted to activate the one or more physical activatable input mechanisms upon being manipulated by a user.

5. The medical visualization system of claim 4, wherein a first communicator element of the one or more communicator elements extends from a first side to a second side, wherein the first side is arranged exteriorly of the handle and the second side is arranged interiorly of the handle, and wherein the second side is adapted to contact a first physical activatable input mechanism of the one or more physical activatable input mechanism when the component coupling part is coupled with the device coupling part.

6. The medical visualization system of claim 1, wherein the transmission component comprises a device cable extending from a first cable end to a second cable end, wherein the component coupling part is provided at the second cable end.

7. The medical visualization system of claim 6, wherein the device cable comprises a stiffened portion extending from the second cable end towards the first cable end.

8. The medical visualization system of claim 7, wherein the stiffened portion has a length of between 20-150 mm.

9. The medical visualization system of claim 1, wherein the component coupling part has a primary coupling element being elongated and extending along a coupling part axis, and wherein the device coupling part has a secondary coupling element forming a cavity adapted to receive the primary coupling element.

10. Medical visualization system according to claim 9, wherein the device coupling part comprises a twist lock adapted to engage by rotation of the handle with a protruding element of the component coupling part when the primary coupling element is fully received by the secondary coupling element.

11. The medical visualization system of claim 10, wherein the stiffened portion comprises a lock sensor configured to transmit a lock signal to the component processor, the lock signal indicating a completed physical connection between the device coupling part and the component coupling part when the primary coupling element is fully received by the secondary coupling element.

12. The medical visualization system of claim 1, further comprising a cable reel adapted to reel in at least a portion of the device cable.

13. The medical visualization system of claim 12, wherein the cable reel comprises a cable reel housing enclosing the portion of the device cable when reeled in.

14. The medical visualization system of claim 13, wherein the cable reel comprises a UV light emitter accommodated in the cable reel housing.

15. The medical visualization system of claim 1, further comprising a support bracket having a distal support bracket part and a proximal support bracket part, and wherein the distal support bracket part includes a coupling part retainer adapted to retain the component coupling part of the transmission component.

16. The medical visualization system of claim 15, wherein the proximal support bracket part is adapted to be coupled to the housing of the video processing apparatus, such that the support bracket follows rotation of the housing around a first axis and does not follow rotation of the housing around a second axis perpendicular to the first axis.

17. The medical visualization system of claim 15, wherein the coupling part retainer is adapted to receive the component coupling part by linear movement of the component coupling part along a third axis, and/or wherein the component coupling part is adapted to engage with the coupling part retainer by linear movement of the component coupling part along the third axis.

18. The medical visualization system of claim 17, wherein the component coupling part has a primary coupling element being elongated and extending along a coupling part axis, wherein the coupling part axis is non-parallel with the third axis when the component coupling part is received by the coupling part retainer.

19. The medical visualization system of claim 17, wherein the component coupling part has a primary coupling element being elongated and extending along a coupling part axis, wherein the coupling part axis is perpendicular to the third axis when the component coupling part is received by the coupling part retainer.

20. The medical visualization system of claim 15, wherein the support bracket comprises the cable reel, the cable reel being driven by an actuator, and wherein the actuator is activated to reel in the portion of the device cable based on a first control input, wherein the coupling part retainer comprises a switch subjective to receipt of the component coupling part and the switch triggers the first control input, and wherein the first control input is triggered by the component coupling part being received by the coupling part retainer.
